(19) United States
(12) Patent Application Publication
(10) Pub. No.: US 2006/0110439 A1
(43) Pub. Date: May 25, 2006

(54) Dermal delivery of n-methyl-glucamine and n-methyl-glucamine compounds

(75) Inventors: Annette Tobia, Wyndmoor, PA (US); Francis Kappler, Philadelphia, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Dynamis Therapeutics, Inc.

(21) Appl. No.: 11/256,291

(22) Filed: Oct. 21, 2005

Related U.S. Application Data
(60) Provisional application No. 60/621,371, filed on Oct. 22, 2004.

(51) Int. Cl. A61K 9/127

(52) U.S. Cl. 424/450

(57) ABSTRACT

The present invention relates to methods and compositions for the treatment of skin-related conditions and disorders. In one aspect, the invention features methods and compositions for the transdermal delivery of compounds for the treatment of skin-related conditions and disorders, wherein the compositions include meglumine and a liposome component.

### CROSS-REFERENCE TO RELATED APPLICATIONS

This application is entitled to priority under 35 U.S.C. § 119(e) to U.S. Provisional Patent Application No. 60/621,371, filed Oct. 22, 2004, which application is incorporated by reference herein in its entirety.

### BACKGROUND OF THE INVENTION

Aging Skin

Skin is the largest organ of the human body covering an area of about 16 square feet. It provides protection from the elements, physical injuries, and provides sensory information. It is the first mammalian defense against invasion by bacteria, viruses, and other toxic elements and acts as an excretory organ, removing toxins from the body via perspiration.

Skin consists of two main layers: the dermis and epidermis. The dermis is the inner layer of skin that contains nerve fibers, fat cells, blood vessels, sweat and oil glands, and hair follicles. The dermis also contains collagen and elastin, two proteins that are responsible for the structure and elasticity of the skin itself. Both of these proteins are subject to the process of aging.

The epidermis is the outermost layer of the skin. New cells generated by the dermis continually replace this layer. The epidermis also contains melanocytes or pigment cells. These cells produce melanin, which determines the shade of the skin.

As humans age, certain changes in the skin can be seen and felt. The skin becomes drier, more wrinkled, less resilient and spots and growths appear. Cuts and abrasions may heal more slowly. Genetically programmed chronologic aging causes biochemical changes in collagen, elastin, and the connective tissues that give skin its firmness and elasticity. The genetic program for each person is different, so the loss of skin firmness and elasticity occurs at different rates and different times in one individual as compared with another. As skin becomes less elastic, it also becomes drier. Underlying fat padding begins to disappear. With loss of underlying support by fat padding and connective tissues, the skin begins to sag. It looks less supple, and wrinkles form.

Simultaneously with genetically programmed aging, the process of photoaging may be taking place. Photoaging is the effect of chronic and excessive sun exposure on the skin. Cigarette smoking also contributes to aging effects by the biochemical changes it brings about in skin tissues. Photoaging interacts with chronologic aging and appears to hasten the process of chronologic aging. In fact, photoaging may be responsible for the majority of age-associated changes in the skin's appearance.

Although the skin provides a painless and compliant interface for systemic drug administration (dermal or transdermal delivery), it is also able to impede the flux of toxins into the body which means that it naturally has a very low permeability to the penetration of foreign molecules (Wertz et al., 1989, Transdermal Drug Delivery: Development Issues and Research Initiatives p. 1-17). A unique hierarchical structure of lipid-rich matrix with embedded corneocytes in the upper strata (15 μm) of skin—the stratum corneum—is responsible for this barrier (Prausnitz et al., 2004, Nat. Rev. Drug Discov. 3:115-124.

3-deoxyglucosone (3DG)

Two of the most dangerous substances to biological macromolecules are the same as those essential for life—oxygen and glucose.

Various harmful forms of oxygen are generated in the body; singlet oxygen, superoxide radicals, hydrogen peroxide, and hydroxyl radicals all cause tissue damage. A catchall term for these and similar oxygen related species is “reactive oxygen species” (ROS). ROS damage tissue proteins, lipids, and nucleic acids (DNA) and are endpoints of many chronic and acute diseases such as cancer, atherosclerosis, diabetes, aging, rheumatoid arthritis, dementia, trauma, stroke, and infection. ROS are also generated from glucose. One mechanism is through the formation of cytotoxic carbonyls, such as methylglyoxal (MG) and 3-deoxyglucosone (3DG) that are known precursors to the formation of Advanced Glycation End Products (AGEs).

An extremely important consequence of AGEs is their binding to receptors on many different types of cells. The best-known receptor is RAGE, which belongs to the immunoglobulin superfamily. The internalization of AGEs by their receptors lead to increased production of ROS in the cell and increased levels of cytokines, endothelin, thrombomodulin and other inflammatory factors. It should be noted that the number of RAGE receptors are increased under conditions of hyperglycemia.

MG production is the result of a mistake in glycolysis and, as such, cannot be controlled therapeutically. The body removes most MG via the glyoxylase pathway, which requires glutathione, a compound that also protects cells from ROS by direct interaction with ROS species. 3DG escapes detoxification by the glyoxylase pathway but is converted to 3-deoxyfructose, an inert metabolite, by aldehyde reductase; however, 3DG can also compromise the activity of this enzyme.

3DG has many toxic effects on cells and is present at elevated concentrations in several disease states. Some of the harmful effects of 3DG with regard to ROS formation and aging are as follows:
3DG induces reactive oxygen species, which results in oxidative DNA damage (Shimoi et al., 2001, Mutat. Res. 480-481:371-378)
3DG inactivates some of the most important enzymes that protect cells from ROS. For example, glutathione peroxidase, a central antioxidant enzyme that uses glutathione to remove ROS, and glutathione reductase, which regenerates glutathione, are both inactivated by 3DG (Vander Jagt et al., 1997, Biochem. Pharmacol. 53:1133-1140; Niwa et al., 2001, Kidney Int. Suppl. 78:S37-S41).
3DG inactivates aldehyde reductase (Takahashi et al., 1995, Biochemistry 34:1433-1438). This is important, since aldehyde reductase is the cellular enzyme that protects the body from 3DG. There is evidence that this detoxification of 3DG to 3-deoxyfructose (3DF) is impaired in diabetic humans since their ratio of urinary and plasma 3DG to 3DF differs significantly from non-diabetic individuals (Lal et al., 1997, Arch. Biochem. Biphys. 342:254-260).
3DG induced reactive oxygen species contribute to the development of diabetic complications (Araki, 1997, Nippon Ronen Igakkai Zasshi 34:716-720). Specifically, 3DG induces heparin-binding epidermal growth factor, a smooth muscle mitogen that is abundant in atherosclerotic plaques. This suggests that an increase in 3DG may trigger atherogenesis in diabetes. (Taniguchi et al., 1996, Diabetes 45 Suppl. 3:S81-83; Che et al., 1997, J. Biol. Chem. 272:18453-18459). Further, the development of diabetic complications is accelerated in patients with extremely high levels of 3DG in their serum (Kusunoki et al., 2003, Diabetes Care 26:1889-94).
3DG is a teratogenic factor in diabetic embryopathy leading to embryo malformation (Eriksson et al., 1998, Diabetes 47:1960-1966). This appears to arise from 3DG accumulation, which leads to superoxide-mediated embryopathy.
3DG induces apoptosis in macrophage-derived cells (Okado et al., 1996, Biochem. Biphys. Res. Commun. 225:219-224) and is toxic to cultured cortical neurons (Kikuchi et al., 1999, J. Neurosci. Res. 57:280-289) and PC12 cells (Suzuki et al., 1998, J. Biochem (Tokyo) 123:353-357). A recent study on the cause of amyotropic lateral sclerosis, a form of motor neuron disease, has suggested that accumulation of 3DG can lead to neurotoxicity as a result of ROS generation (Shinpo et al., 2000, Brain Res. 861:151-159).
   3DG and Aging Skin

3DG glycates and crosslinks protein leading to a complex mixture of compounds called advanced glycation end products (AGEs) (Baynes et al., 1987, Methods Enzymol. 106:88-98; Dyer et al., 1991, J. Biol. Chem. 266:11654-11660). AGEs form as a natural consequence of aging and are implicated in many inflammatory diseases such as diabetes, atherosclerosis, and dementia. AGEs are most commonly formed on long-lived structural proteins such as collagen type I, which is a major structural component of the skin. Crosslinking is a major component of the genetically programmed biochemical changes in collagen, elastin, and the connective tissues that is observed in chronologically aged skin. Importantly, 3DG is found in human skin.

Fructosamine-3-Kinase (F3K)

As reviewed by Brownlee, the previously generally accepted pathway for formation of 3DG comprises a reversible reaction between glucose and the ε-NH₂ groups of lysine-containing proteins, forming a Schiff base (Brownlee, 1994, Diabetes 43:836-841). This Schiff base then rearranges to form a more stable ketoamine known as fructose-lysine (FL) or the “Amadori product”. The dogma has been that 3DG production resulted exclusively from subsequent non-enzymatic rearrangement, dehydration, and fragmentation of the fructoselysine containing protein (Brownlee, 1994, Diabetes 43:836-841). Recent work has shown that an enzymatic pathway for the production of 3DG exists (see FIGS. 1 and 2 and Brown et al., U.S. Pat. No. 6,004,958).

A metabolic pathway was discovered that produces relatively high concentrations of 3DG in organs affected by diabetes (Brown et al., U.S. Pat. No. 6,004,958); and more recently it was found that the pathway also exists in the skin. It was found that a specific kinase (fructosamine-3-kinase, or Amadorase) converts fructose-lysine into fructose-lysine-3-phosphate (FL3P) in an ATP dependent reaction, and that FL3P then breaks down to form free lysine, inorganic phosphate, and 3DG. Brown et al., U.S. Pat. No. 6,004,958, describe a class of compounds that inhibit the enzymatic conversion of fructose-lysine to FL3P and inhibit thereby formation of 3DG. Specific compounds that are representative of the class have also been described (Brown et al., International Publication No. WO 98/33492). For example, it was found that urinary or plasma 3DG can be reduced by meglumine (N-methyl glucamine), sorbitollysine, mannitollysine, and galactitollysine. Id. It was also found that diets high in glycated protein are harmful to the kidney and cause a decrease in birth rate. Id. It has also been disclosed that the fructose-lysine pathway is involved in kidney carcinogenesis. Id. Further, previous studies demonstrate that diet and 3DG can play a role in carcinogenesis associated with this pathway (see International Publication Nos. WO 00/24405, WO 00/62626, and WO 98/33492).

There exists a need to provide methods and compositions for reducing the levels of and the production of toxic and harmful substances, such as 3DG, in living organisms, in order to improve health and longevity. In particular, there is a need to provide inhibitors of fructosamine-3-kinase and inactivators of 3DG, and a need to identify methods of delivering such compounds simply and efficiently, to treat skin aging, inflammatory skin disorders, and to relieve pain, among other things. The present invention meets these needs.

### SUMMARY OF THE INVENTION

The invention includes a dermally-acting composition for application to the skin, comprising a delivery vehicle and meglumine. The invention also includes a dermally-acting composition for application to the skin, comprising a liposome component and meglumine.

In the invention, meglumine can be a hydrochloride salt. Additionally, a composition can further comprise arginine, or a hydrochloride salt thereof. Moreover, a composition can further comprise salicylic acid, or a penetration-enhancing compound, or any combination thereof. Further still, a composition of the invention may comprise a derivative of meglumine, arginine, salicylic acid, or any combination thereof.

A composition of the invention can include delivery vehicle that is a liposome component such as NATIPIDE II, BIPHASIX, and NANOSOMES. A composition of the invention can also include a non-liposome component delivery vehicle such as PHOSAL or PHOSPHOLIPON.

A dermally-acting composition of the invention may further comprise at least one additional substance such as, but not limited to, water, oil, wax, squalene, myristate, triglycerides, cocoa butter, shea butter, alcohol, stearate, a chelating agent, propylene glycol, SEPIGEL, silicone, a silicone derivative, a vitamin, and an amino acid, or any combination thereof.

In a particular embodiment, a dermally-acting composition for application to the skin can include 0.01%-35% delivery vehicle and 0.001%-30% meglumine, and additionally, 0%-30% arginine.

The invention also includes a method for reducing the level of 3-deoxyglucosone (3DG) in the skin of a mammal, comprising contacting the skin of said mammal with a dermally-acting composition, wherein the composition comprises a delivery vehicle and meglumine. In one aspect, the delivery vehicle comprises a liposome component. In another aspect, the composition further comprises arginine. In yet another aspect, the composition further comprises salicylic acid.

The invention also includes a method for reducing the level of 3-deoxyglucosone (3DG) in the skin of a mammal to treat skin aging or skin wrinkling, comprising contacting the skin of said mammal with a dermally-acting composition, wherein the composition comprises a delivery vehicle and meglumine. In one aspect, the delivery vehicle comprises a liposome component. In another aspect, the composition further comprises arginine. In yet another aspect, the composition further comprises salicylic acid.

The invention also includes a method for reducing the level of 3-deoxyglucosone (3DG) in the skin of a mammal to prevent skin aging or skin wrinkling, comprising contacting the skin of said mammal with a dermally-acting composition, wherein the composition comprises a delivery vehicle and meglumine. In one aspect, the delivery vehicle comprises a liposome component. In another aspect, the composition further comprises arginine. In yet another aspect, the composition further comprises salicylic acid.

The invention also includes a method for reducing the level of 3-deoxyglucosone (3DG) in the skin of a mammal to treat pain, comprising contacting the skin of said mammal with a dermally-acting composition, wherein the composition comprises a delivery vehicle and meglumine. In one aspect, the delivery vehicle comprises a liposome component. In another aspect, the composition further comprises arginine. In yet another aspect, the composition further comprises salicylic acid.

The invention also includes a method for reducing the level of 3-deoxyglucosone (3DG) in the skin of a mammal to treat an inflammatory disorder, comprising contacting the skin of said mammal with a dermally-acting composition, wherein the composition comprises a delivery vehicle and meglumine. In one aspect, the delivery vehicle comprises a liposome component. In another aspect, the composition further comprises arginine. In yet another aspect, the composition further comprises salicylic acid. In an aspect, the inflammatory disorder can be eczema, psoriasis, rosacea, and radiation-induced dermatitis.

The invention also includes a method for reducing the level of 3-deoxyglucosone (3DG) in the skin of a mammal to treat itch, comprising contacting the skin of said mammal with a dermally-acting composition, wherein the composition comprises a delivery vehicle and meglumine. In one aspect, the delivery vehicle comprises a liposome component. In another aspect, the composition further comprises arginine. In yet another aspect, the composition further comprises salicylic acid. In an aspect, the itch can be cutaneous itch, neuropathic itch, neurogenic itch, mixed-type itch, and psychogenic itch.

The invention also includes a kit for reducing the level of 3-deoxyglucosone (3DG) in the skin of a mammal, wherein the kit comprises a dermally-acting composition for application to the skin, comprising a delivery vehicle and meglumine, an applicator and instructions for the use of the kit. In one aspect, the delivery vehicle includes a liposome component. In another aspect, the kit further includes as least one additional compound, such as arginine or salicylic acid.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, the drawings illustrate various embodiments of the invention. It should be understood, however, that the invention is not limited to the precise arrangements and instrumentalities shown. In the drawings:

FIG. 1 is a schematic drawing depicting the non-enzymatic production of 3DG.

FIG. 2 is a schematic drawing which illustrates the activity of fructosamine kinase. Fructose-lysine (FL) is phosphorylated by a fructosamine kinase such as Amadorase to form fructoselysine-3-phosphate (FL3P). FL3P spontaneously decomposes into lysine, Pi and 3DG (Brown et al., U.S. Pat. No. 6,004,958).

FIG. 3 is a schematic illustration of both protein adduct formation by 3DG and inhibition of protein adduct formation by 3DG. 3DG can form an adduct with a primary amino group on a protein by way of a Schiff base, the equilibrium of which is depicted. The protein-3DG Schiff base adduct may go on to form a crosslinked protein, through the formation of a second protein-3DG adduct by way of the 3DG molecule involved in the first protein-3DG Schiff base adduct described above, thereby forming a “3DG bridge” between two primary amino groups of a single protein or two different proteins (pathway “A”). The first protein-3DG Schiff base adduct may be prevented from going on to form such crosslinked proteins as depicted in pathway “B”. For example, such protein crosslinking may be inhibited by nucelophilic agents such as glutathione (GSH) or penicillamine. Such nucleophilic agents react with the 3DG carbon atom responsible for forming the second Schiff base, preventing that carbon atom from forming a Schiff base protein-3DG adduct and thereby preventing crosslinking of the protein.

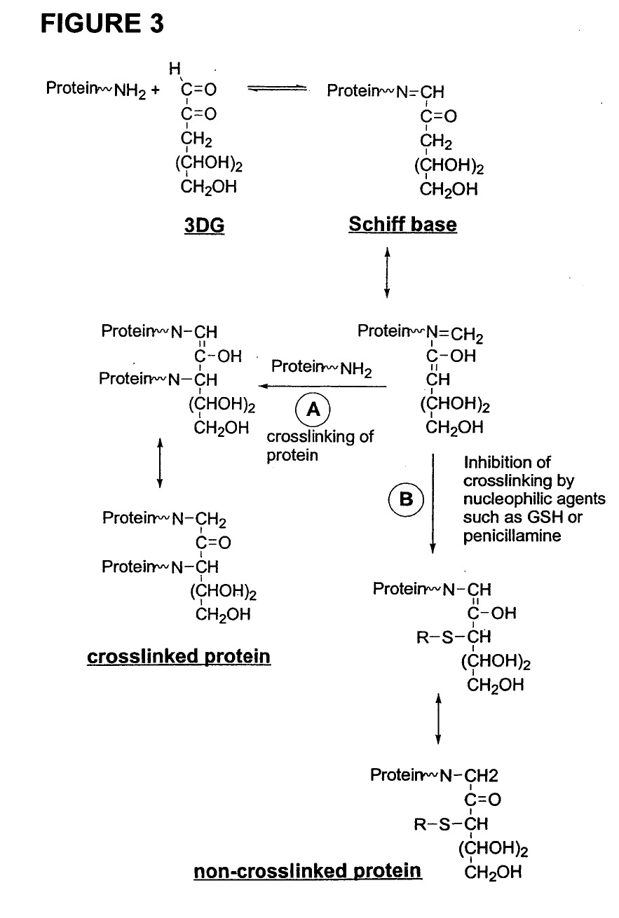

FIG. 4 is an image of an electrophoretic gel depicting the effect of inactivating 3DG using arginine on the 3DG-dependent crosslinking of collagen type I.

FIG. 5 is an image of an agarose gel showing DNA products of an RT-PCR reaction using kidney and skin cDNAs and F3K oligonucleotide primers.

FIG. 6 is a graph depicting the average erythema scores as determined by an expert grader of human volunteers' SLS-treated skin after treatment with either (i) a base cream (Cream A), (ii) a base cream containing meglumine-HCl and arginine (Cream B) or (iii) with no treatment.

FIG. 7 is a graph depicting the average erythema scores measured with a chromameter of human volunteers' SLS-treated skin after treatment with either (i) a base cream (Cream A), (ii) a base cream containing meglumine-HCl and arginine (Cream B) or (iii) with no treatment.

FIG. 8 is a graph depicting the average transdermal evaporative water loss (TEWL) of human volunteers' SLS-treated skin after treatment with either (i) a base cream (Cream A), (ii) a base cream containing meglumine-HCl and arginine (Cream B) or (iii) with no treatment.

### DETAILED DESCRIPTION OF THE INVENTION

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “accumulation of 3DG” or “accumulation of alpha-dicarbonyl sugars” as used herein refers to a detectable increase in the level of 3DG and/or alpha-dicarbonyl sugar over a period of time.

“Alpha-dicarbonyl sugar,” as used herein, refers to a family of compounds, including 3-deoxyglucosone, glyoxal, methyl glyoxal and glucosone.

“Alpha-dicarbonyl sugar associated parameter of wrinkling, aging, disease or disorder of the skin,” as used herein, refers to the biological markers described herein, including 3DG levels, 3DF levels, fructosamine kinase levels, protein crosslinking, and other markers or parameters associated with alpha-dicarbonyl sugar associated wrinkling, aging, diseases or disorders of the skin.

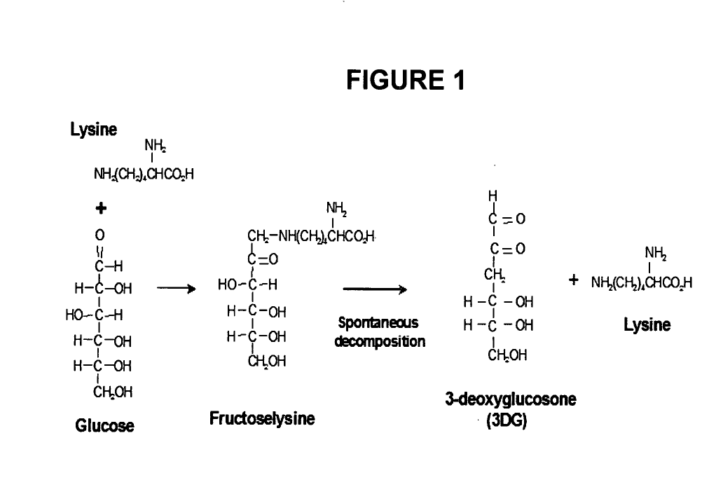

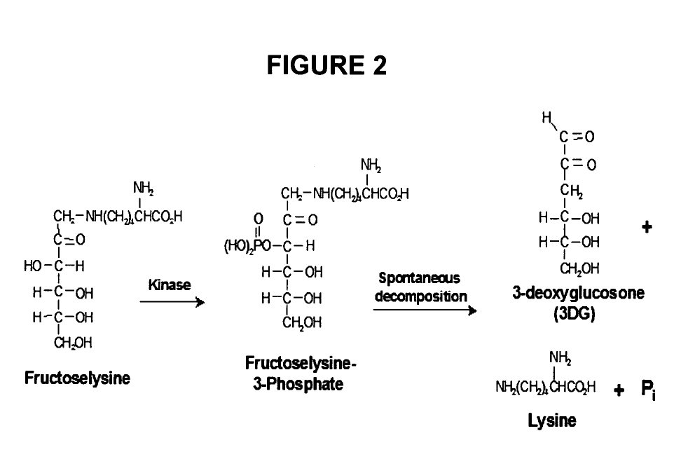

“3-deoxyglucosone” or “3DG,” as used herein, refers to the 1,2-dicarbonyl-3-deoxysugar (also known as 3-deoxyhexulosone), which can be formed via an enzymatic pathway or can be formed via a non-enzymatic pathway. For purposes of the present description, the term 3-deoxyglucosone is an alpha-dicarbonyl sugar which can be formed by pathways including the non-enzymatic pathway described in FIG. 1 and the enzymatic pathway resulting in breakdown of FL3P or F3P described in FIG. 2. Another source of 3DG is diet. 3DG is a member of the alpha-dicarbonyl sugar family, also known as 2-oxoaldehydes.

A “3DG associated” or “3DG related” disease or disorder as used herein, refers to a disease, condition, or disorder which is caused by, indicated by, or associated with 3DG, including defects related to enhanced synthesis, production, formation, and accumulation of 3DG, as well as those caused by, medicated by or associated with decreased levels of degradation, detoxification, binding, and clearance of 3DG. Similarly, a “glyoxal-related” disorder, a “methyl glyoxal related” disorder, a “glucosone-related” disorder, and an “alpha dicarbonyl sugar-related’ disorder refers to a disorder caused by or associated with each of the respective compounds.

“A 3DG inhibiting amount” or an “alpha-dicarbonyl inhibiting amount” of a compound refers to that amount of compound that is sufficient to inhibit the function or process of interest, such as synthesis, formation accumulation and/or function of 3DG or another alpha-dicarbonyl sugar.

“3-O-methyl sorbitollysine (3-O-Me-sorbitollysine),” is an inhibitor of fructosamine kinases, as described herein. It is used interchangeably with the term “DYN 12”.

The term “AGE-proteins” (Advanced Glycation End product modified proteins), as used herein, refers to a product of the reaction between sugars and proteins (Brownlee, 1992, Diabetes Care 15:1835; Niwa et al., 1995, Nephron 69:438). For example, the reaction between protein lysine residues and glucose, which does not stop with the formation of fructose-lysine (FL). FL can undergo multiple dehydration and rearrangement reactions to produce non-enzymatic 3DG, which reacts again with free amino groups, leading to cross-linking and browning of the protein involved. AGEs also include the products that form from the reaction of 3DG with other compounds, such as lipids and nucleic acids.

Fructosamine-3-kinase, F3K, fructosamine phosphokinase, fructosamine-3-phosphokinase (FN3K) collectively or individually “Amadorase” is responsible for the production of 3DG. More specifically, it refers to a protein which can enzymatically convert fructoselysine to fructoselysine-3-phosphate or fructose to fructose-3-phosphate when additionally supplied with a source of high energy phosphate.

The term “Amadori product,” as used herein, refers to a ketoamine, such as, but not limited to, fructoselysine, comprising a rearrangement product following glucose interaction with the ε-NH₂ groups of lysine-containing proteins.

As used herein, “amino acids” are represented by the full name thereof, by the three-letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biological sample,” as used herein, refers to samples obtained from a living organism, including skin, hair, tissue, blood, plasma, cells, sweat and urine.

The term “clearance,” as used herein refers to the physiological process of removing a compound or molecule, such as by diffusion, exfoliation, removal via the bloodstream, and excretion in urine, or via other sweat or other fluid.

A “compound,” as used herein, refers to any type of substance or agent that is commonly considered a drug, or a candidate for use as a drug, as well as combinations and mixtures of the above, or modified versions or derivatives of the compound.

“Detoxification” of 3DG refers to the breakdown or conversion of 3DG to a form that does not allow it to perform its normal function. Detoxification can be brought about or stimulated by any composition or method, including “pharmacologic detoxification”, or metabolic pathway that can cause detoxification of 3DG.

“Pharmacologic detoxification of “3DG” or other alpha-dicarbonyl sugars refers to a process in which a compound binds with or modifies 3DG, which in turn causes it to be become inactive or to be removed by metabolic processes such as, but not limited to, excretion.

A “disease” is a state of health of an animal wherein the animal cannot maintain homeostasis, and wherein if the disease is not ameliorated then the animal's health continues to deteriorate. As used herein, normal aging is included as a disease.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” or “therapeutically effective amount” of a compound is that amount of compound which is sufficient to provide a beneficial effect to the subject to which the compound is administered, or gives the appearance of providing a therapeutic effect as in a cosmetic.

As used herein, the term “effector domain” refers to a domain capable of directly interacting with an effector molecule, chemical, or structure in the cytoplasm which is capable of regulating a biochemical pathway.

The term “formation of 3DG” refers to 3DG which is not necessarily formed via a synthetic pathway, but can be formed via a pathway such as the spontaneous or induced breakdown of a precursor.

The term “fructose-lysine” (FL) is used herein to signify any glycated-lysine, whether incorporated in a protein/peptide or released from a protein/peptide by proteolytic digestion. This term is specifically not limited to the chemical structure commonly referred to as fructose-lysine, which is reported to form from the reaction of protein lysine residues and glucose. As noted above, lysine amino groups can react with a wide variety of sugars. Indeed, one report indicates that glucose is the least reactive sugar out of a group of sixteen (16) different sugars tested (Bunn et al., 1981, Science, 213:222). Thus, tagatose-lysine formed from galactose and lysine, analogously to glucose is included wherever the term fructose-lysine is mentioned in this description, as is the condensation product of all other sugars, whether naturally-occurring or not. It will be understood from the description herein that the reaction between protein-lysine residues and sugars involves multiple reaction steps. The final steps in this reaction sequence involve the crosslinking of proteins and the production of multimeric species, known as AGE-proteins, some of which are fluorescent. Once an AGE protein forms, then proteolytic digestion of such AGE-proteins does not yield lysine covalently linked to a sugar molecule. Thus, these species are not included within the meaning of “fructose-lysine”, as that term is used herein.

The term “fructose-lysine-3-phosphate,” as used herein, refers to a compound formed by the enzymatic transfer of a high energy phosphate group from ATP to FL. The term fructose-lysine-3-phosphate (FL3P), as used herein, is meant to include all phosphorylated fructose-lysine moieties that can be enzymatically formed whether free or protein-bound.

The term “fructose-3-phosphate,” as used herein, refers to a compound formed by the enzymatic transfer of a high-energy phosphate group from ATP to Fructose. The term fructose-3-phosphate (F3P), as used herein, is meant to include all phosphorylated fructose moieties that can be enzymatically formed.

“Fructoselysine-3-phosphate kinase” (FL3K), as used herein, refers to one or more proteins, such as Amadorase, which can enzymatically convert FL to FL3P or enzymatically convert Fructose to F3P, as described herein, when supplied with a source of high energy phosphate. The term is used interchangeably with “fructoselysine kinase (FLK)”, fructosamine kinase, fructosamine phosphokinase, fructosamine-3-kinase (F3K), fructosamine-3-phosphokinase (FN3K), and with “Amadorase”.

The term “FL3P Lysine Recovery Pathway,” as used herein, refers to a lysine recovery pathway which exists in human skin and kidney, and possibly other tissues, and which regenerates unmodified lysine as a free amino acid or as incorporated in a polypeptide chain.

The term “glycated diet,” as used herein, refers to any given diet in which a percentage of normal protein is replaced with glycated protein. The expressions “glycated diet” and “glycated protein diet” are used interchangeably herein.

“Glycated lysine residues,” as used herein, refers to the modified lysine residue of a stable adduct produced by the reaction of a reducing sugar and a lysine-containing protein. The majority of protein lysine residues are located on the surface of proteins as expected for a positively charged amino acid. Thus, lysine residues on proteins, which come in contact with serum, or other biological fluids, can freely react with sugar molecules in solution. This reaction occurs in multiple stages. The initial stage involves the formation of a Schiff base between the lysine free amino group and the sugar keto-group. This initial product then undergoes the Amadori rearrangement, to produce a stable ketoamine compound.

This series of reactions can occur with various sugars. When the sugar involved is glucose, the initial Schiff base product will involve imine formation between the aldehyde moiety on C-1 of the glucose and the lysine ε-amino group. The Amadori rearrangement will result in formation of lysine coupled to the C-1 carbon of fructose, 1-deoxy-1-(ε-aminolysine)-fructose, herein referred to as fructose-lysine or FL. Similar reactions will occur with other aldose sugars, for example galactose and ribose (Dills, 1993, Am. J. Clin. Nutr. 58:S779). For the purpose of the present invention, the early products of the reaction of any reducing sugar and the ε-amino residue of protein lysine are included within the meaning of glycated-lysine residue, regardless of the exact structure of the modifying sugar molecule.

The term “induction of 3DG” or “inducing 3DG,” as used herein, refers to methods or means which start or stimulate a pathway or event leading to the synthesis, production, or formation of 3DG or increase in its levels, or stimulate an increase in function of 3DG. Similarly, the phrase “induction of alpha-dicarbonyl sugars”, refers to induction of members of the alpha-dicarbonyl sugar family, including 3DG, glyoxal, methyl glyoxal, and glucosone.

The term “inflammatory skin disorders” refers to skin conditions characterized by redness, inflammation, tenderness, scaling, and/or itch. Such disorders include psoriasis, eczema, rosacea, skin itch due to uremia, and radiation induced dermatitis.

“Inhibiting 3DG” as described herein, refers to any method or technique that inhibits 3DG synthesis, production, formation, accumulation, or function, as well as methods of inhibiting the induction or stimulation of synthesis, formation, accumulation, or function of 3DG. It also refers to any metabolic pathway that can regulate 3DG function or induction. The term also refers to any composition or method for inhibiting 3DG function by detoxifying 3DG or causing the clearance of 3DG. Inhibition can be direct or indirect. Induction refers to induction of synthesis of 3DG or to induction of function. Similarly, the phrase “inhibiting alpha-dicarbonyl sugars”, refers to inhibiting members of the alpha-dicarbonyl sugar family, including 3DG, glyoxal, methyl glyoxal, and glucosone.

The term “inhibiting accumulation of 3DG,” as used herein, refers to the use of any composition or method which decreases synthesis, increases degradation, or increases clearance, of 3DG such that the result is lower levels of 3DG or functional 3DG in the tissue being examined or treated, compared with the levels in tissue not treated with the composition or method. Similarly, the phrase “inhibiting accumulation of alpha-dicarbonyl sugars”, refers to inhibiting accumulation of members of the alpha-dicarbonyl sugar family, including 3DG, glyoxal, methyl glyoxal, and glucosone, and intermediates thereof.

As used herein, an “instructional material” includes a publication, a recording, a diagram, or any other medium of expression that can be used to communicate the usefulness of the peptide of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound invention or be shipped together with a container which contains the identified compound. Alternatively, the instructional material can be shipped separately from the container with the intention that the instructional material and the compound be used cooperatively by the recipient.

“Modified” compound, as used herein, refers to a modification or derivation of a compound, which may be a chemical modification, such as in chemically altering a compound in order to increase or change its functional ability or activity.

The term “mutagenicity” refers to the ability of a compound to induce or increase the frequency of mutation. The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequences (i.e., A, T, G, C), this also includes an RNA sequence (i.e., A, U, G, C) in which “U” replaces “T.”

The term “peptide” typically refers to short polypeptides.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

A “polynucleotide” means a single strand or parallel and anti-parallel strands of a nucleic acid. Thus, a polynucleotide may be either a single-stranded or a double-stranded nucleic acid.

A “prophylactic” treatment is a treatment administered to a subject who does not exhibit signs of a disease or exhibits only early signs of the disease for the purpose of decreasing the risk of developing pathology associated with the disease.

The term “protein” typically refers to large polypeptides.

“Reactive oxygen species” refers to various harmful forms of oxygen that are generated in the body; singlet oxygen, superoxide radicals, hydrogen peroxide, and hydroxyl radicals are examples of such molecules that cause tissue damage. A catchall term for these and similar oxygen related species is “reactive oxygen species” (ROS). The term also includes ROS formed by the internalization of AGEs into cells and the ROS that form therefrom.

The terms “removing 3-deoxyglucosone” and “reducing the level of 3-deoxyglucosone,” as used herein, refers to any composition or method, the use of which results in lower levels of 3-deoxyglucosone (3DG) or lower levels of functional 3DG when compared to the level of 3DG or the level of functional 3DG in the absence of the composition. Lower levels of 3DG can result from its decreased synthesis or formation, increased degradation, increased clearance, or any combination of thereof. Lower levels of functional 3DG can result from modifying the 3DG molecule such that it can function less efficient in the process of glycation or can result from binding of 3DG with another molecule which blocks and/or inhibits the ability of 3DG to function. Lower levels of 3DG can also result from increased clearance and excretion in urine of 3DG. The term is also used interchangeably with “inhibiting accumulation of 3DG”. Similarly, the phrase “removing alpha-dicarbonyl sugars”, refers to removal of members of the alpha-dicarbonyl sugar family, including 3DG, glyoxal, methyl glyoxal, and glucosone.

Also, the terms glycated-lysine residue, glycated protein and glycosylated protein or lysine residue are used interchangeably herein, is consistently with current usage in the art where such terms are art-recognized used interchangeably.

The term “skin,” as used herein, refers to the commonly used definition of skin, e.g., the epidermis and dermis, and the cells, glands, mucosa and connective tissue that comprise the skin.

The term “skin wrinkling” refers to the development of fine lines such as those around the eyes and mouth, upper arm, neck, chest and deep brow furrows. The term “skin aging” refers to changes in tone, color (yellowing), texture, and moisture (dryness) of the skin.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not limited to, a purified marker of interest which has been labeled, such as with a radioactive isotope, allowing it to be distinguished from an endogenous substance in a sample.

A “susceptible test animal,” as used herein, refers to a strain of laboratory animal which, due to for instance the presence of certain genetic mutations, have a higher propensity toward a disease disorder or condition of choice, such as diabetes, cancer, and the like.

“Synthesis of 3DG”, as used herein refers to the formation or production of 3DG. 3DG can be formed based on an enzyme dependent pathway or a non-enzyme dependent pathway. Similarly, the phrase “synthesis of alpha-dicarbonyl sugars”, refers to synthesis or spontaneous formation of members of the alpha-dicarbonyl sugar family, including 3DG, glyoxal, methyl glyoxal, and glucosone, and adducts as disclosed herein.

A “therapeutic” treatment is a treatment administered to a subject who exhibits signs of pathology, for the purpose of diminishing or eliminating those signs.

By “transdermal” delivery is intended both transdermal (or “percutaneous”) and transmucosal administration, i.e., delivery by passage of a drug through the skin or mucosal tissue and into the bloodstream. Transdermal also refers to the skin as a portal for the administration of drugs or compounds by topical application of the drug or compound thereto.

The term “topical application”, as used herein, refers to administration to a surface, such as the skin. This term is used interchangeably with “cutaneous application”.

As used herein, the term “liposome” refers to a microscopic, fluid-filled structure, with walls comprising one or more layers of phospholipids and molecules similar in physical and/or chemical properties to those that make up mammalian cell membranes, such as, but not limited to, cholesterol, stearylamine, or phosphatidylcholine. Liposomes can be formulated to incorporate a wide range of materials as a payload either in the aqueous or in the lipid compartments.

As used herein, the term “dermal” refers to the skin, and in particular, the thickness of the skin from outer, dead layer, down to the bottom of the skin in direct contact with the inside of the body.

“Dermal delivery” of a substance refers to delivery of that substance into the skin, and preferably, at least into the outer, epidermal layer of skin, and more preferably, into the lower, dermal layer of skin. Therefore, “dermal delivery” of a substance refers to contacting the skin with the substance, wherein the substance penetrates at least the outermost layer of the skin. The term also refers to the delivery of the substance to additional layers of the skin, including, but not limited to, delivery of the substance all of the way down to the bottom layer in the skin in direct contact with the inside of the body.

A substance is said to be “dermally-acting” when the substance acts either on or within the skin, or both. A dermally-acting substance is not precluded from crossing the skin (i.e., “transdermal delivery”) and entering the inside of the body (eg., the systemic blood circulation), although the substance may or may not enter the inside of the body.

As used herein, the term, “transdermal delivery vehicle” indicates a composition comprising at least one first compound that can facilitate transdermal delivery of at least one second compound associated with, or in close physical proximity to, the composition comprising the first compound.

Similarly, a “dermal delivery vehicle” refers to a composition comprising at least one first compound that can facilitate dermal delivery of at least one second compound associated with, or in close physical proximity to, the composition comprising the first compound.

The term “delivery vehicle” is used herein as a generic reference to any delivery vehicle, including, but not limited to, dermal delivery vehicles and transdermal delivery vehicles.

The term “phospholipids” refers to any member of a large class of fatlike organic compounds that in their molecular structure resemble the triglycerides, except for the replacement of a fatty acid with a phosphate-containing polar group. One end of the molecule is soluble in water (hydrophilic) and water solutions. The other, fatty acid, end is soluble in fats (hydrophobic). In watery environments, phospholipids naturally combine to form a two-layer structure (lipid bilayer) with the fat-soluble ends sandwiched in the middle and the water-soluble ends sticking out. Such lipid bilayers are the structural basis of cell membranes and liposomes.

The term “sonophoresis” refers to the use of ultrasound to permeabilize skin for a prolonged period of time for the purpose of delivering compounds through the skin into the body or to allow for the sampling of interstitial fluid or its components.

The term “electroporation” refers to the transitory structural perturbation of lipid bilayer membranes due to the application of short duration, high voltage pulses for the purpose of enhancing the delivery of compounds through the skin in to the body.

The term “iontophoresis” refers to the use of a long duration low-density electrical current that attracts the ions in the compound to be delivered drives them through the skin.

The terms “permeation enhancement” and “permeation enhancers” as used herein relate to the process and added materials which bring about an increase in the permeability of skin to a poorly skin permeating pharmacologically active agent, i.e., so as to increase the rate at which the drug permeates through the skin and enters the bloodstream. “Permeation enhancer” is used interchangeably with “penetration enhancer.”

The term to “treat,” as used herein, means reducing the frequency with which symptoms are experienced by a patient or subject or administering an agent or compound to reduce the frequency and/or severity with which symptoms are experienced. As used herein, “alleviate” is used interchangeably with the term “treat.”

As used herein, “treating a disease, disorder or condition” means reducing the frequency or severity with which a symptom of the disease, disorder or condition is experienced by a patient. Treating a disease, disorder or condition may or may not include complete eradication or elimination of the symptom.

### DETAILED DESCRIPTION

The invention is based in part on the discovery that the penetration of N-methyl glucamine compounds, and preferably, meglumine, into at least the first layer of skin has a beneficial effect on the skin. This is because it has been demonstrated herein for the first time that meglumine, and compositions comprising meglumine, when delivered to the skin, have the effect of treating, soothing, or improving the skin, and/or treating diseases and disorders of the skin. Such compositions of the invention minimally include meglumine and a dermal delivery vehicle.

The present invention is also based in part, on the discovery that compounds that inhibit the enzyme fructosamine-3-kinase, and further, compounds that inactivate 3DG, can treat and/or prevent skin aging and skin wrinkling. It has been discovered that when the compounds of the invention are administered according to methods of the present invention in a liposome formulation, the beneficial effects of such compounds are enhanced compared with the administration of the compounds in the absence of liposome formulations.

Fructosamine-3-kinase is known to be present and active in the skin, and 3DG is known to exist in the skin, as disclosed in WO 05/079463 and WO 03/089601, each of which is incorporated herein by reference in its entirety. Also disclosed in WO 05/079463 and WO 03/089601 are methods and compositions for inhibiting fructosamine-3-kinase, and for inactivating 3DG. Meglumine is one such compound that is useful for inhibiting fructosamine-3-kinase, and for inactivating 3DG. However, it is shown herein for the first time that novel compositions comprising meglumine and liposomes are synergistically effective at inhibiting fructosamine-3-kinase, and for inactivating 3DG in the skin.

The present invention therefore features novel dermally-acting compositions for the treatment and prevention of skin aging, skin wrinkling, skin-associated pain, skin irritation and inflammation, and itch. As described in detail herein, the compositions of the invention comprise meglumine and a delivery vehicle, and in a preferred embodiment, meglumine and liposomes. However, compositions of the invention can also include compounds that further enhance the beneficial effects of mixtures of meglumine and a delivery vehicle, including, but not limited to, arginine and salicylic acid.

The invention also features methods for the dermal delivery of F3K inhibitors and 3DG inactivators to relieve pain. The invention further encompasses methods using delivery vehicles to deliver meglumine-based compositions to the skin, including liposome-mediated methods of delivery of N-methyl-glucamine compounds, alone or in combination with 3DG inactivators such as arginine, penicillamine, aminoguanidine, creatine, n-acetylcysteine, or other molecules that contain guanidine or biguanide groups, to the skin in order to treat inflammatory skin conditions, reduce skin aging, and to reduce pain. Further still, the invention includes methods of administering a dermally-acting composition of the invention for the treatment of itch.

Compositions for Dermal Delivery

There are several advantages to delivering compounds, including cosmetics, drugs or other therapeutic agents, into the skin (dermal drug delivery) or into the body through the skin (transdermal drug delivery). Transdermal compound delivery offers an attractive alternative to injections and oral medications. Dermal compound delivery offers an efficient way to deliver a compound to the skin of a mammal, and preferably a human, and provides a method of treatment of the skin, or otherwise provides a method of affecting the skin, without the need to break or damage the outer layer of the skin. In the present invention, dermal delivery, by way of a dermally-acting compound of the invention, provides these advantages for treatment of a skin-related condition, disorder or disease.

A number of compounds, including some drugs, will penetrate the skin effectively. Nicotine, estrogen, scopolamine, fentanyl, and nitroglycerine are among the few drugs that can be successfully delivered transdermally from patches simply because the molecules are relatively small and potent at small doses of 0.1 mg to 15 mg/day (Kanikkannan et al., 2000, Curr. Med. Chem. 7:593-608). Many other compounds and drugs can be delivered only when an additional enhancement system is provided to “force” them to pass through the skin. Among several methods of transdermal drug delivery are electroporation, sonophoresis, iontophoresis, permeation enhancers (cyclodextrins), and liposomes. While the aforementioned methods are also included in the present invention for dermal delivery of the compounds of the invention, liposomes represent a preferred dermal delivery method.

In one aspect of the invention, a dermally-acting composition is provided for treatment of 3DG-related conditions in the skin, wherein the composition comprises an N-methyl glucamine compound and a delivery vehicle. In one aspect, a dermally-acting composition is provided for treatment of 3DG-related conditions in the skin, wherein the composition comprises an N-methyl glucamine compound and a liposome component. In an embodiment, the N-methyl glucamine compound is meglumine. In another embodiment, the meglumine is a hydrochloride salt. In yet another embodiment, the N-methyl glucamine compound is a meglumine derivative.

In another aspect of the invention, a dermally-acting composition is provided for treatment of 3DG-related conditions in the skin, wherein the composition comprises an N-methyl glucamine compound, arginine, and a delivery vehicle. In another aspect, a dermally-acting composition is provided for treatment of 3DG-related conditions in the skin, wherein the composition comprises an N-methyl glucamine compound, arginine, and a liposome component. In an embodiment, the arginine is a hydrochloride salt. In yet another embodiment, the arginine is an arginine derivative.

In yet another aspect of the invention, a dermally-acting composition is provided for treatment of 3DG-related conditions in the skin, wherein the composition comprises an N-methyl glucamine compound, salicylic acid, and delivery vehicle. In an aspect, a dermally-acting composition is provided for treatment of 3DG-related conditions in the skin, wherein the composition comprises an N-methyl glucamine compound, salicylic acid, and a liposome component. In one embodiment, the composition further comprises arginine.

An obstacle for topical administration of compounds in general, and in particular for pharmaceuticals, is the stratum corneum layer of the epidermis. The stratum corneum is a highly resistant layer comprised of protein, cholesterol, sphingolipids, free fatty acids and various other lipids, and includes cornified and living cells. One of the factors that limits the penetration rate (flux) of a compound through the stratum corneum is the amount of the active substance which can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

The invention encompasses the preparation and use of a dermally-acting composition comprising a compound useful for treatment of various skin related diseases, disorders, or conditions described herein, including skin aging, photoaging, and wrinkling of the skin. Such a composition may consist of the active ingredient alone, in a form suitable for administration to a subject, or the composition may comprise at least one active ingredient and one or more pharmaceutically acceptable carriers, one or more additional ingredients, or some combination of these. The active ingredient may be present in the composition in the form of a physiologically acceptable ester or salt, such as in combination with a physiologically acceptable cation or anion, as is well known in the art. Compositions of the invention will also be understood to encompass pharmaceutical compositions useful for treatment of other conditions, disorders and diseases associated with the skin.

The formulations of the compositions described herein may be prepared by any method known or hereafter developed in the art. Similarly, the formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Therefore, in one aspect, a dermal delivery vehicle of the invention is a composition comprising at least one first compound that can facilitate dermal delivery of at least one second compound associated with, or in close physical proximity to, the composition comprising the first compound. As will be understood by the skilled artisan, when armed with the disclosure set forth herein, such delivery vehicles include, but should not be limited to, liposomes, nanosomes, phosopholipid-based non-liposome compositions (eg., selected cochleates), among others. Other non-limiting examples of delivery vehicles useful in the present invention include PHOSAL (eg., phospholipids) and PHOSPHOLIPON (phospholipid fraction) (American Lecithin Company, Oxford, Conn.), as well as BIPHASIX (Helix BioPharma Corp., Aurora, ON) and NANOSOMES (L'Oreal USA, New York, N.Y.).

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

A composition of the invention may be prepared, packaged, or sold in bulk, as a single unit dose, or as a plurality of single unit doses. As used herein, a “unit dose” is a discrete amount of the composition comprising a predetermined amount of the active ingredient, including a dermally-acting ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the carrier, and any additional ingredients in a composition of the invention will vary, depending upon the identity, size, and condition of the subject treated and further depending upon the route by which the composition is to be administered. By way of example, the composition may comprise between 0.001% and 99.9% (w/w) active ingredient.

In addition to the active ingredient, a composition of the invention may further comprise one or more additional pharmaceutically active agents. Particularly contemplated additional agents include anti-emetics and scavengers such as cyanide and cyanate scavengers.

Controlled- or sustained-release formulations of a composition of the invention may be made using conventional technology, in addition to the disclosure set forth elsewhere herein. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the compositions of the invention.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, nanoparticles, or microspheres or a combination thereof that facilitates the controlled-release of the active ingredient.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 0.001% to about 90% (w/w) active ingredient, although the concentration of the active ingredient may be as high as the solubility limit of the active ingredient in the solvent. Formulations for topical administration may further comprise one or more of the additional ingredients described herein.

In one aspect of the invention, a dermal delivery system includes a liposome composition. By way of a non-limiting example, a liposome delivery system useful in the present invention is commercially available from KUHS GmbH+Co. Laboratories under the trade name NATIPIDE II, which liposome systems are prepared under U.S. Pat. No. 5,741,513. However, it will be understood, based on the disclosure set forth herein, that any liposome delivery system may be useful in the present invention, and that the present invention should not be construed to be limited to any particular liposome delivery system. That is, based on the disclosure set forth herein, the skilled artisan will understand how to identify a liposome delivery system as being useful in the present invention. By way of a non-limiting example, a liposome delivery system that can facilitate dermal delivery of meglumine, such that the delivery of meglumine results in the inhibition of 3DG production, or in the inactivation of 3DG, is a liposome delivery system useful in the present invention.

The present invention also encompasses the improvement of dermal and transdermal drug delivery through the use of penetration enhancers (also called sorption promoters or accelerants), which penetrate into skin to reversibly decrease the barrier resistance. Many compounds are known in the art for penetration enhancing activity, including sulphoxides (such as dimethylsulphoxide, DMSO), azones (e.g. laurocapram), pyrrolidones (for example 2-pyrrolidone, 2P), alcohols and alkanols (ethanol, or decanol), glycols (for example propylene glycol, PG, a common excipient in topically applied dosage forms), surfactants (also common in dosage forms) and terpenes. Other enhancers include oleic acid, oleyl alcohol, ethoxydiglycol, laurocapram, alkanecarboxylic acids, dimethylsulfoxide, polar lipids, or N-methyl-2-pyrrolidone.

Many potential sites and modes of action have been identified for skin penetration enhancers; the intercellular lipid matrix in which the accelerants may disrupt the packing motif, the intracellular keratin domains or through increasing drug partitioning into the tissue by acting as a solvent for the permeant within the membrane. Further potential mechanisms of action, for example with the enhancers acting on desmosomal connections between corneocytes or altering metabolic activity within the skin, or exerting an influence on the thermodynamic activity/solubility of the drug in its vehicle are also feasible (Williams et al., 2004, Adv. Drug Deliv. Rev. 56:603-618).

In another aspect, cyclodextrins are cyclic oligosaccharides with a hydrophilic outer surface and a somewhat lipophilic central cavity. Cyclodextrins are able to form water-soluble inclusion complexes with many lipophilic water-insoluble drugs. In aqueous solutions, drug molecules located in the central cavity are in a dynamic equilibrium with free drug molecules. Furthermore, lipophilic molecules in the aqueous complexation media will compete with each other for a space in the cavity. Due to their size and hydrophilicity only insignificant amounts of cyclodextrins and drug/cyclodextrin complexes are able to penetrate into lipophilic biological barriers, such as intact skin. In general, cyclodextrins enhance topical drug delivery by increasing the drug availability at the barrier surface. At the surface, the drug molecules partition from the cyclodextrin cavity into the lipophilic barrier. Thus, drug delivery from aqueous cyclodextrin solutions is both diffusion controlled and membrane controlled. It appears that cyclodextrins can only enhance topical drug delivery in the presence of water (Loftsson et al., 2001, Int. J. Pharm. 225:15-30).

In alternative embodiments, the topically active pharmaceutical or cosmetic composition may be optionally combined with other ingredients such as moisturizers, cosmetic adjuvants, anti-oxidants, chelating agents, bleaching agents, tyrosinase inhibitors and other known depigmentation agents, surfactants, foaming agents, conditioners, humectants, wetting agents, emulsifying agents, fragrances, viscosifiers, buffering agents, preservatives, sunscreens and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art.

In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art. The compositions of this invention may also contain active amounts of retinoids (i.e., compounds that bind to any members of the family of retinoid receptors), including, for example, tretinoin, retinol, esters of tretinoin and/or retinol and the like.

The topically active pharmaceutical or cosmetic composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. More preferable, it should be present in an amount from about 0.0005% to about 5% of the composition; most preferably, it should be present in an amount of from about 0.001% to about 1% of the composition. Such compounds may be synthetically- or naturally-derived.

Liquid derivatives and natural extracts made directly from biological sources may be employed in the compositions of this invention in a concentration (w/v) from about 1 to about 99%. Fractions of natural extracts and protease inhibitors may have a different preferred rage, from about 0.01% to about 20% and, more preferably, from about 1% to about 10% of the composition. Of course, mixtures of the active agents of this invention may be combined and used together in the same formulation, or in serial applications of different formulations.

The composition of the invention may comprise a preservative from about 0.005% to 2.0% by total weight of the composition. The preservative is used to prevent spoilage in the case of an aqueous gel because of repeated patient use when it is exposed to contaminants in the environment from, for example, exposure to air or the patient's skin, including contact with the fingers used for applying a composition of the invention such as a therapeutic gel or cream. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound for use in the invention in the aqueous gel formulation. Preferred antioxidants for some compounds are BHT, BHA, alpha-tocopherol and ascorbic acid in the preferred range of about 0.01% to 5% and BHT in the range of 0.01% to 1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefor as would be known to those skilled in the art.

Therefore, in an exemplary embodiment of the invention, a compound of the invention includes a delivery vehicle, meglumine, and at least one additional component, such as an emulsifier, a penetration-enhancing compound, a preservative, or a binding agent, among others, or a combination of two or more such compounds in addition to a delivery vehicle and meglumine. In one aspect, a delivery vehicle is a liposome component. Additional components include, but should not be limited to those including water, oil (eg., olive oil/PEG7, evening primrose oil), biovera oil, wax (eg., jojoba wax), squalene, myristate (eg., isopropyl myristate), triglycerides (eg., caprylic triglyceride), Solulan 98, cocoa butter, shea butter, alcohol (eg., behenyl alcohol), stearate (eg., glycerolmonostearate), chelating agents (eg., EDTA), propylene glycol, SEPIGEL (Seppic, Inc., Fairfield, N.J.), silicone and silicone derivatives (eg., dimethicone, cyclomethicone), vitamins (eg., vitamin E), and amino acids (eg., arginine), among others.

The invention also encompasses a composition of matter comprising a phospholipid or a liposome and N-methyl-glucamine compounds either alone or in combination with 3DG inactivators such as arginine, penicillamine, aminoguanidine, creatine, n-acetylcysteine, or other molecules that contain guanidine or biguanide groups.

The invention also encompasses a composition of matter as described herein to include skin lighteners such as tyrosinase inhibitors, arbutin, kojic acid, and ascorbic acid (vitamin C), exfoliants such α and β-hydroxy-acid, L-carnitine, glycolic acid, or salicylic acid, and/or preservatives such as acids (benzoic, salicylic) alcohols (benzyl, ethyl), paraben (butyl, ethyl), isothiazolinones (benzisothiazolinone), formalydehyde releasers (diazolidinyl urea, imidazaolidinal urea), and other materials (iodopropynl butylcarbamate, sodium hydroxymethylglycinate) in phospholipids or liposomes.

The invention further encompasses a composition of matter as described herein to include topical itch treatments, including, but not limited to, antihistamines and corticosteroids.

It will be understood, based on the disclosure set forth herein, that any of the components of a composition of the invention, as discussed herein or later discovered, can be used at varying concentrations in the composition, based on the purpose and compatibility of the component within the composition. Unless otherwise described herein, and by way of a non-limiting example, a component of a composition of the invention can be used at varying concentrations ranging from 0.001%-50%, as measured by weight or by volume of the entire composition. In other embodiments, a component of a composition of the invention can be used in a range of 0.005%-40%, a range of 0.01%-30%, a range of 0.05%-20%, a range of 0.1%-10%, and a range of 0.5%-5%. A component of a composition of the invention can also be used at a level of 0.1%, 0.5%, 1%, 1.5%, 2%, 2.5%, 3%, 4%, and 5%. Other concentrations and ranges of concentrations can be used and will be understood to be within the scope of the invention, based on the disclosure set forth herein.

Liposome Compositions of the Invention

Liposomes comprise a preferred composition of the present invention in combination with an N-methyl-glucamine compound. In one aspect of the invention, a dermally-acting composition of the invention comprises meglumine and a liposome component. Based on the disclosure set forth herein, the skilled artisan will understand the additional components that can be added to a liposome/meglumine composition of the present invention, for the purpose of treating or preventing skin wrinkling, skin aging, skin irritation or inflammation, pain and itch.

Liposomes are microscopically small, hollow phospholipid spheres, which can be composed of one or several concentrically arranged phospholipid double membranes. Liposomes can be loaded with a variety of substances. Lipophilic active substances dissolve in the bilayer, amphiphilic substances become associated with the phospholipid membrane and hydrophilic substances occur in solution in the enclosed aqueous volume (Artmann et al., 1990, Drug Res. 40 (II) Nr. 12 pp. 1363-1365). Liposomes used as drug carriers or for topical cosmetic use are non-toxic and available in industry (Gehring et al., 1990, Drug Res. 40 (II) Nr. 12, pp. 1368-1371).

Often liposomes are distinguished by their number of lamellae and size. Small unilamellar vesicles are surrounded by one membrane and have diameters of 20 nm to 100 nm while large unilamellar vesicles range up to one micron. Multilamellar vesicles consist of several concentric membrane layers and range up to several microns (Presentation by J. Roding at workshop “Liposomes and Skin” May 5, 1990 Paris entitled “Characterization of Liposomes and NATIPIDE II System”).

Liposomes can be formed from a variety of natural membrane components, such as cholesterol stearylamine, or phosphatidylcholine, and can be formulated to incorporate a wide range of materials as a payload either in the aqueous or in the lipid compartments. See, for example, U.S. Pat. Nos. 5,120,561 and 6,007,838, each of which are incorporated herein by reference in their entirety.

The versatility of liposomes, due to the variable composition, enables liposomes to be used to deliver vaccines, proteins, nucleotides, plasmids, drugs, or cosmetics to the body. Liposomes can be used as carriers for lipophilic drugs like the anti-tumor and the anti-viral derivatives of azidothymidine (AZT) (Kamps, et al., 1996, Biochim. Biophys. Acta. 1278:183-190). Insulin can also be delivered via liposomes (Muramatsu et al., 1999, Drug Dev. Ind. Pharm. 25:1099-1105). For medical uses as drug carriers, the liposomes can also be injected intravenously and when they are modified with lipids, their surfaces become more hydrophilic and hence the circulation time in the bloodstream can be increased significantly. So-called polyethylene glycol modified “stealth” liposomes are especially being used as carriers for hydrophilic (water soluble) anti-cancer drugs like doxorubicin. Liposomal derivatives of mitoxantrone and others are especially effective in treating diseases that affect the phagocytes of the immune system because they tend to accumulate in the phagocytes, which recognize them as foreign invaders (Rentsch et al., 1997, Br. J. Cancer 75:986-992). They have also been used to carry normal genes into a cell to treat diseases caused by defective genes (Guo et al., 2000, Biosci. Rep. 20:419-432).

Liposomes are also sometimes used in cosmetics because of their moisturizing qualities. Phospholipids combined with water immediately formed a sphere because one end of each molecule is water soluble, while the opposite end is water insoluble. There are several known process for making multilamellar liposome-encapsulated material on an industrial scale. Rao, “Preparation of Liposomes on the Industrial Scale: Problems and Perspectives,” in LIPOSOME TECHNOLOGY, Vol. I, G. Gregordias, ed., (CRC Press, 1984) pp. 247-257. In the most widely used of these, a thin lipid film (from an organic solvent) is deposited on the walls of a container, an aqueous solution of the material to be encapsulated is added, and the container is agitated (Bangham et al., 1965, J. Mol. Biol. 13:238). Under the right conditions, this simple process, results in the formation of multilamellar vesicles of liposomes trapping the material. Success of this procedure relies heavily on the formation of the thin lipid film, and variation in encapsulation is seen with different methods of agitation. However, the skilled artisan will understand that any method of making liposomes to form a composition of the present invention can be useful, and should be considered to be within the scope of the invention.

Liposome compositions of the invention can comprise any range of liposome and meglumine components as identified as useful, according to the methods and detailed description set forth herein. By way of a non-limiting example, a liposome component of a composition of the invention may include from 0.001% to 99.9% liposome component, or more preferably, from 0.1%-50% liposome component, and even more preferably, from 0.1%-30% liposome component.

However, the invention also includes compositions including non-liposome delivery vehicles, either alone, or in combination with a liposome delivery vehicle. A composition of the invention can comprise any range of delivery vehicle and meglumine components as identified as useful, according to the methods and detailed description set forth herein. Therefore, it will also be understood that a delivery vehicle in a composition of the invention may include from 0.001% to 99.9% delivery vehicle, or more preferably, from 0.1%-50% delivery vehicle, and even more preferably, from 0.1%-30% delivery vehicle.

Methods of Delivering 3DG Inhibitors and Inactivators

The present invention features, in part, a method of inhibiting an enzyme which is involved in the enzymatic synthetic pathway of 3DG production, wherein the enzyme is expressed in skin (for example, see Experimental Example 2 below). Furthermore, because it has also been discovered in the present invention that 3DG is present at high levels in skin (see Experimental Example 3 below), the invention also features methods of dermal delivery of meglumine/liposome compositions to a mammal in order to inhibit and/or inactivate at least one of the routes of 3DG production in the skin, including enzymatic synthesis, non-enzymatic synthesis and non-enzymatic formation of 3DG. In yet another embodiment, the present invention features methods which interfere with the function of 3DG in skin. The mammal is preferably a human.

In one embodiment, the invention features a method for reducing the level of 3DG in the skin of a mammal. In one aspect of the invention, the mammal is a human. In an embodiment of the invention, the method includes contacting the skin of a mammal with a dermally-acting composition comprising meglumine and a delivery vehicle. In another embodiment of the invention, the method includes contacting the skin of a mammal with a dermally-acting composition comprising meglumine and a liposome component. Vehicles useful for transdermal delivery according to the invention include, but should not be limited to, liposomes, as well as penetration-enhancing compounds. As will be understood by the disclosure set forth herein, combinations of two or more compounds that mediate and/or enhance transdermal delivery are also included in the present invention.

Based on the disclosure set forth herein, it will be understood that a compound useful for inhibiting the production or activity of 3DG is useful in a delivery vehicle-based composition of the present invention, and furthermore, in a liposome-based composition of the present invention. By way of a non-limiting example, such compounds include, but are not limited to, those disclosed and discussed in WO 05/079463 and WO 03/089601, each of which is incorporated herein by reference in its entirety. In one embodiment, a compound that inhibits production of 3DG is an N-methyl glucamine compound. In another embodiment, the compound that inhibits production of 3DG is meglumine.

In another embodiment, the invention features a method for reducing the level of 3DG in the skin of a mammal. In an embodiment of the invention, the method includes contacting the skin of a mammal with a dermally-acting composition comprising meglumine, a delivery vehicle, and at least one additional compound. In one aspect, a delivery vehicle is a liposome component. In one embodiment, the composition further comprises arginine. In another embodiment, the composition further comprises salicylic acid. Other components useful in a dermally-acting composition of the invention are set forth in detail elsewhere herein.

In an embodiment of the invention, a method of reducing the level of 3DG in a mammal includes contacting the skin of a mammal with a composition comprising meglumine and a delivery vehicle, and the method further comprises using a transdermal delivery method to deliver an inhibitory or inactivating compound to the mammal transdermally. In one aspect, a delivery vehicle is a liposome. Transdermal delivery methods useful in the invention include, but should not be limited to, iontophoresis, electroporation, and sonophoresis, among others. In one embodiment, a compound that inactivates 3DG is an N-methyl glucamine compound. In another embodiment, the compound is meglumine.

It is a feature of the present invention, therefore, to treat conditions, disorders or diseases associated with the skin. Such disorders include, but should not be limited to, itch (eg., cutaneous itch, neuropathic itch, neurogenic itch, mixed-type itch, psychogenic itch), pain and inflammation (eg., eczema, psoriasis, rosacea, radiation-induced dermatitis).

It is also a feature of the present invention to treat a condition, disorder, or disease associated with 3DG in a mammal, wherein the condition, disorder or disease is not directly associated with the skin. The skilled artisan will understand that the transdermal delivery of a compound can enable the delivery of such compounds sub-dermally, such as to joints below or near the skin through which transdermal delivery of a compound occurs. Therefore, in one aspect of the invention, transdermal delivery of an inhibitor or inactivator of 3DG is also useful to treat disorders including, but not limited to, pain and inflammation, such as those types associated with joints, bones, and the musculature.

Kits

The invention also encompasses kits for inhibiting or inactivating 3DG, and for treating 3DG-associated skin diseases and disorders. The invention should be construed to include kits for alpha-dicarbonyl sugars other than 3DG, as well.

In an embodiment, the invention includes a kit comprising composition including an inhibitor of 3DG and a delivery vehicle, and an instructional material which describes the administration of the composition to a mammal. In one embodiment, the inhibitor is meglumine. In another embodiment, the composition further comprises arginine. In yet another embodiment, the delivery vehicle is a liposome. The invention should be construed to include other embodiments of kits that are known to those skilled in the art, such as a kit comprising a standard and a (preferably sterile) solvent suitable for dissolving or suspending the composition of the invention prior to administering the compound to a cell or an animal. Preferably the animal is a mammal. More preferably, the mammal is a human.

In another embodiment, the invention includes a kit comprising composition including an inhibitor of 3DG and a liposome component, and an instructional material which describes the administration of the composition to a mammal. In one embodiment, the inhibitor is meglumine. In another embodiment, the composition further comprises arginine. The invention should be construed to include other embodiments of kits that are known to those skilled in the art, such as a kit comprising a standard and a (preferably sterile) solvent suitable for dissolving or suspending the composition of the invention prior to administering the compound to a cell or an animal. Preferably the animal is a mammal. More preferably, the mammal is a human.

In yet another embodiment, the invention includes a kit comprising composition including an inactivator of 3DG and a liposome component, and an instructional material which describes administering the composition to a mammal. In one embodiment, the inactivator is meglumine. In another embodiment, the composition further comprises arginine. In yet another embodiment, the composition comprises salicylic acid.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

### Inhibition of 3DG Collagen Crosslinking In Vitro

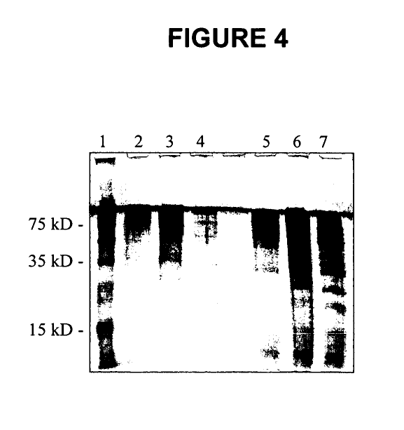

The direct inactivation of 3DG is a method of reducing 3DG levels. Calf skin collagen type 1 (1.3 mg) was incubated with no addition, with 5 mM 3DG, or with 5 mM 3DG plus 10 mM of arginine for 24 hr. Each sample was digested with cyanogen bromide (CnBr) to create peptide fragments that are visualized by sodium-dodecyl sulfate-polyacrylamide gel electrophoresis (SDS-PAGE) (FIG. 4). Lane 2 is collagen alone; lane 3 is collagen plus 5 mM 3DG; and lane 4 is collagen plus 5 mM 3DG plus 10 mM arginine. Lanes 5, 6, and 7 are the same, but with twice as much sample applied.

Crosslinking was assessed by visually determining the amount of high molecular weight protein remaining near the origin of the resolving gel, as compared to the amount that migrates into the gel matrix. The more crosslinking that exists, the more material there is near the origin of the gel. The lanes containing collagen with 3DG (#3, #6) have more material residing at the origin than those containing collagen alone (#2, #5). Lanes containing 3DG plus arginine (#4, #7) show that arginine was able to inactivate 3DG and prevent it from crosslinking collagen.

### Example 2

### Localization of Amadorase mRNA in Skin

The presence of Amadorase mRNA was analyzed and was utilized as one measure of the ability of skin to produce the 3DG present in skin. PolyA+ messenger RNA isolated from human kidney and skin was obtained from Stratagene. The mRNA was used in RT-PCR procedures. Using the published sequence for human Amadorase (Delpierre et al., 2000, Diabetes 49:1627-1634; Szwergold et al., 2001, Diabetes 50:2139-2147), a reverse primer to the 3′ terminal end of the gene (bp 930-912) was used in a reverse transcriptase reaction to create a cDNA template for subsequent PCR. This same primer was used along with a forward primer from the middle of the Amadorase gene (bp412-431) to amplify a 519 bp fragment. Human skin and kidney samples were subjected to RT-PCR and analyzed by agarose gel electrophoresis, as were controls which contained no cDNA templates.

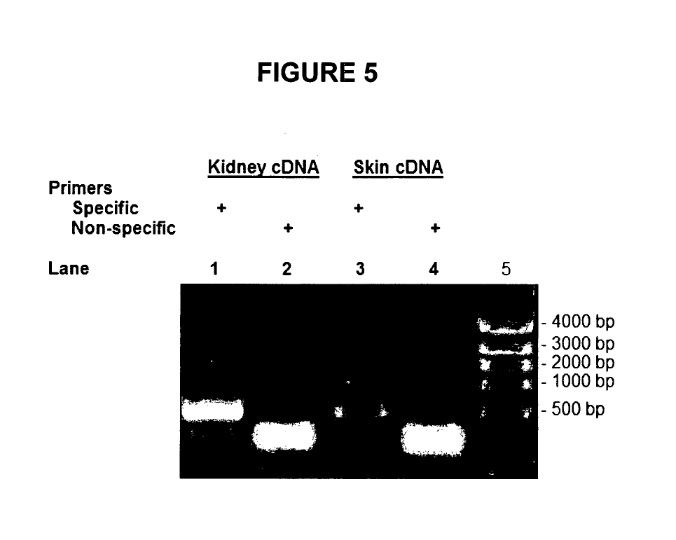

A 519 bp product, evidence of Amadorase mRNA was found in both kidney and skin; no such product was seen in the samples which received no cDNA template. (FIG. 5, lanes 2 and 4). The results demonstrate that skin expresses Amadorase mRNA. Subsequent translation of the protein would account for production of 3DG in skin.

### Example 3

### Localization of 3DG in Skin

One centimeter (1 cm) squares of skin from six mice were prepared and subjected to extraction with perchloric acid. 3DG was derivatized with a 10-fold excess of diaminonapthalene in PBS. Ethyl acetate extraction provided a salt-free fraction which was converted to the trimethyl silyl ether with Tri-Sil (Pierce). Analysis was performed on a Hewlett-Packard 5890 selected ion monitoring GC-MS system GC was performed on a fused silica capillary column (Hewlett-Packard DB-5 column measuring 25 m×0.25 mm) using the following temperature program: injector port 250° C. at 16° C./minute and held for 15 minutes. Quantitation of 3DG employed selected ion monitoring using an internal standard of U-¹³C-3DG.

The average amount of 3DG detected in the skin was 1.46±0.3 μM. This value was substantially higher than the plasma concentrations of 3DG detected in the same animals (0.19±0.05 μM). These data indicate that the high levels of 3DG in the skin are due to production or accumulation of 3DG in the skin.

### Example 4

### Formulation of a Liposome Cream Delivery System

23.9 grams of BIOCREME Concentrate from BioChemica International Inc., was blended with 2.9 grams cocoa butter, 1.4 grams shea butter, 2.2 grams aloe oil, 1.1 grams vitamin E, 3.7 grams glycerol, 51 grams water, 1.1 grams dimethicone and 10.8 grams NATIPIDE II containing 1 gram arginine-HCl and 1 gram meglumine-HCl.

TABLE 4
Chest Cream (used in all studies except psoriasis study)

| Component | weight percentage |
|---|---|
| Part A | |
| Olive oil PEG 7 | 1 |
| Evening Primrose Oil | 1 |
| Biovera Oil | 2 |
| Jojoba Wax | 3 |
| Squalene | 2 |
| Isopropylmyristate | 3 |
| Capric/caprylic triglyceride | 1 |
| SOLULAN 98 | 2 |
| Cocoa Butter | 4 |
| Shea Butter | 2 |
| Behenyl alcohol | 3 |
| Glycerol monostearate | 2 |
| Part B | |
| Water | to 100% |
| EDTA | 0.05 |
| Tetrasodium EDTA | 0.05 |
| Propylene Glycol | 5 |
| SEPIGEL 305 | 5 |
| Part C | |
| Dimethicone, 50 cts | 1 |
| Cyclomethicone | 2 |
| Vitamin E-acetate | 1.5 |
| Vitamin E | 0.5 |
| PHENONIP | 0.3 |
| GERMALL PLUS | 0.3 |
| COSMOPERINE | 1 |
| Meglumine Hydrochloride | 1-5 |
| Arginine hydrochloride | 1-5 |
| Part D | |
| NATIPIDE mix 1 | 10-30 |
| (0-20% meglumine hydrochloride, | |
| 0-20% arginine hydrochloride and | |
| NATIPIDE II to 100%) | |
| Scent | 0.2 |

Part A was melted and mixed with Part B, then homogenized. The first six components of Part C were added and then COSMOPERINE, meglumine-hydrochloride and arginine hydrochloride were added one at a time, with homogenization after each addition. Part D was added using an overhead stirrer.

### Example 5

### Wound Healing Trial

A trial with human volunteers compared the wound-healing properties of a topical preparation as described in Example 4 (Cream B) to a base cream lacking meglumine-HCl and arginine (Cream A). Six sites on the volar forearms (3 on each arm) of 15 female volunteers were exposed on Day 0 to an irritant solution (0.5% sodium lauryl sufate, SLS) under occlusion for 18-24 hr. On Day 1, the four arm sites with the most similar degree of damage for 12 of the volunteers who experienced a significant irritation effect from the SLS were selected for the treatment phase of the study. Patches were removed and panelists then had the test creams applied to the four selected sites twice daily for 7 days. The other forearm sites were not treated so they could be used as controls.

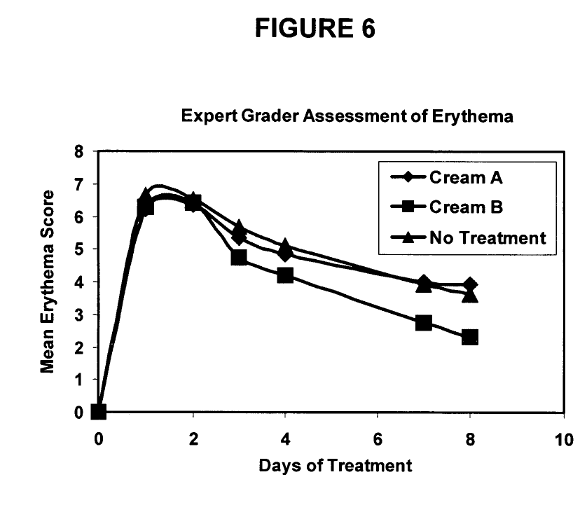

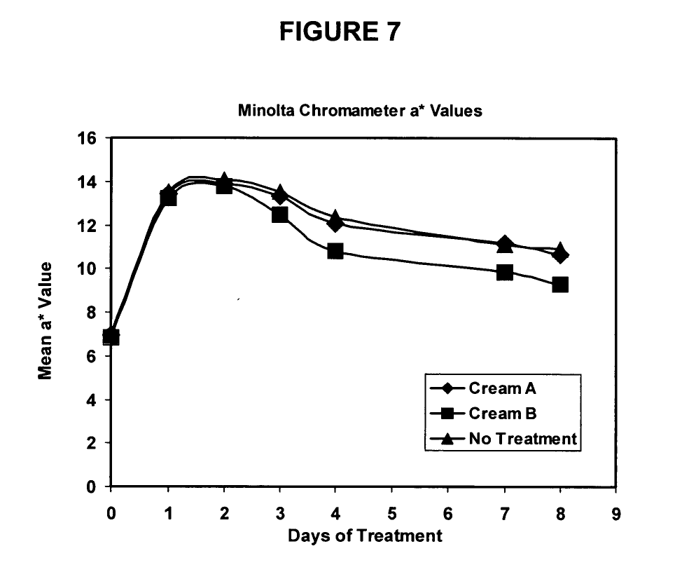

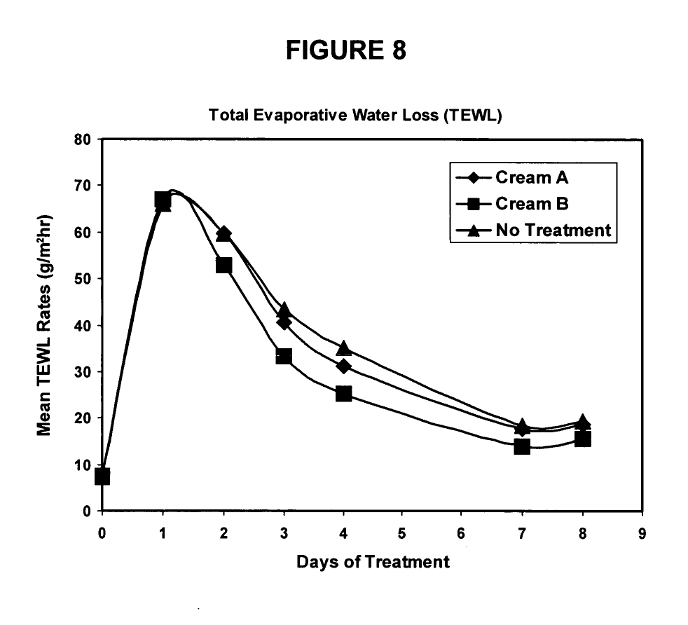

The extent of irritation and healing rates were based on clinical observations of an Expert Grader for erythema (using a 10 point scale), instrument measurements using a Minolta Chromameter (to measure redness) and DermaLab Meter (to measure Transdermal Evaporative Water Loss (TEWL)) on day 0 (prior to SLS exposure), and on days 1, 2, 3, 4, 7, and 8. FIGS. 6 and 7 show the average values for assessments of erythema (redness), and FIG. 8 shows the average values for total evaporative water loss (TEWL) at days 1, 2, 3, 4, 7 and 8 after SLS treatment.

These study results demonstrate that Cream B enhanced the repair of detergent damaged skin. Although there were no clear cut differences in the early stages of the study, from Day 3 onward there were significant differences between Cream A and Cream B. Cream B was more effective in reducing erythema especially with regard to visual assessments being made by the Expert Grader (FIG. 6). It was also determined that Cream B enhanced the restoration of the stratum corneum barrier which had been disrupted by exposure to SLS more than Cream A (FIG. 8).

### Example 6

### Eczema Study

A six year old female child with eczema at multiple skin sites since birth used a cream as in Example 4 containing meglumine-HCl and arginine-HCl. After seven days of daily application of the cream, the symptoms of dryness and itch were diminished.

### Example 7

### Psoriasis Study

A blinded study was conducted with 22 adult volunteers having 2-10% of their body surface area affected with psoriasis. Between 2 and 6 psoriasis-affected sites for each volunteer were chosen for treatment and only one type of cream was used on each volunteer. The volunteers were divided into 3 groups, and the affected sites were treated with twice daily applications of one of the following creams: (1) a base cream containing salicylic acid (1.9%) (“Cream SA”, 7 volunteers); (2) a base cream containing salicylic acid (1.9%) and meglumine hydrochloride (5.5%) and arginine hydrochloride (3.8%)(“Cream SAMA”, 7 volunteers); or (3) a base cream containing meglumine hydrochloride (5.5%) and arginine hydrochloride (3.8%) (“Cream MA”, 8 volunteers) (Table 5).

An expert grader was used to examine the skin areas. Assessments were made at the beginning of the study and after 6 weeks with respect to:

A. Erythema (0=no redness, 1=faint redness, 2=red coloration, 3=very bright red coloration, 4=deep red coloration);

B. Dryness (0=no dryness/scaling, 1=fine scale partially covering lesions, 2=fine to coarse scale covering most or all of the lesions, 3=coarse, non-tenacious scale predominates, covering most or all of the lesions, 4=coarse, thick, tenacious scale over most or all lesions, rough surface);

C. Induration (0=no evidence of plaque elevation, 1=slight but definite plaque elevation, typically edges indistinct or sloped, 2=moderate plaque elevation with rough or sloped edges, 3=marked plaque elevation typically with hard or sharp edges, 4=very marked plaque elevation typically with hard sharp edges); and

D. Pruritis (0=no itching, 1=slightly bothersome itching, 2=bothersome itching, but no loss of sleep, 3=constant itching causing intense discomfort and loss of sleep).

The mean values for the expert grader's scores at 0 weeks (beginning of study) and after 6 weeks are shown in Table 1. A statistical t-test was used to determine the significance of any difference between the means, and underlined values indicate p<0.05. The volunteers treated with the Cream SA exhibited a statistical improvement with respect to all features measured. The volunteers treated with the Cream MA exhibited a statistical benefit for erythema, dryness, and induration. The volunteers treated with the Cream SAMA (salicylic acid with meglumine hydrochloride and arginine hydrochloride) exhibited a statistical benefit for erythema, and dryness and unexpectedly showed substantially greater improvement for pruritis compared to creams containing salicylic acid or meglumine hydrochloride and arginine hydrochloride.

TABLE 1
Results of Psoriasis study for a 6-week treatment period.

| Cream | 0 week | 6 week | p value | 0 week | 6 week | p value |
|---|---|---|---|---|---|---|
| | Erythema | | | Dryness/Scaling | | |
| SA | 1.77 | 1.27 | 0.001 | 2.60 | 1.83 | 0.001 |
| SAMA | 1.76 | 1.45 | 0.033 | 2.28 | 1.76 | 0.064 |
| MA | 2.03 | 1.38 | 0.00001 | 2.03 | 1.45 | 0.001 |
| | Induration | | | Pruritis | | |
| SA | 2.03 | 1.38 | 0.003 | 0.70 | 0.27 | 0.010 |
| SAMA | 1.72 | 1.54 | 0.375 | 0.83 | 0.21 | 0.001 |
| MA | 1.66 | 0.97 | 0.004 | 0.41 | 0.26 | 0.125 |

Mean scores for erythema, dryness/scaling, induration and pruritis for volunteers at 0 and after 6 weeks of treatment.
Results of a statistical t-test are shown;
p values that are <0.05 are underlined.

### Example 8

### Crepy Skin Study

A double-blind trial with human volunteers compared the anti-photoaging effect of a cream containing meglumine-HCl and arginine-HCl (Cream D) as described in Example 4 to a base cream lacking them (Cream C). The creams were tested for their ability to improve skin smoothness, texture and overall appearance after a 4 week treatment. Eighteen female volunteers with moderate photodamage and dryness on the lateral aspect of the upper arm and volar forearm were treated with twice daily applications of both creams (one for each arm) for 4 weeks. Expert graders assessed the visual texture (crepiness), dryness and roughness of the treated areas at the beginning of the study and after the 4 week period. Each skin feature was graded on a scale with 0 being skin that is smooth, firm, resilient, moisturized and 8 being skin that is markedly rough, inflexible, and wrinkled.

Table 3 shows the averaged Expert Graders' assessments of volunteers' skin treated with each cream (a lower number is better) for three features. The D cream significantly reduced (p<0.05) visual dryness and crepiness compared with the base cream (Cream C). Cream D also reduced tactile dryness, with a statistical significance of p<0.10. This study shows that a cream containing meglumine-HCl and arginine-HCl improves the appearance and texture of photo-aged skin.

TABLE 3
Change in skin conditions over time

| | Week 0 | | Week 4 | | Grade Change | | |
|---|---|---|---|---|---|---|---|
| | C | D | C | D | C | D | |
| Skin Feature | Cream | Cream | Cream | Cream | Cream | Cream | t-test |
| Texture (Crepiness) | 5.0 | 5.0 | 4.1 | 3.7 | −.09 | −1.3 | P = 0.032 |
| Visual Dryness | 2.9 | 2.8 | 1.8 | 1.0 | −1.1 | −1.8 | P = 0.006 |
| Tactile Dryness | 3.9 | 4.0 | 2.6 | 2.1 | −1.3 | −1.9 | P = 0.051 |

### Example 9

### Skin Wrinkling Study

A 90 year old female with wrinkled skin on the forearm was treated with a cream as in Example 4 or an identical cream lacking liposomes and containing meglumine-hydrochloride and arginine. The cream prepared with liposomes showed a greater improvement in skin appearance with diminished lines and increased softness.

### Example 10

### Skin Wrinkling Study

A 62 year old female with facial wrinkles used a cream prepared as in Example 4. After several weeks of daily application her skin was smoother, more moisturized, and showed fewer fine lines.

### Example 11

### Pain Study

A 64 year old male with tension or sinus headaches applied a cream prepared as in Example 4 to the forehead and sinus areas of the face. The headache pain diminished after application of the cream.

### Example 12

### Pain Study

A 90 year old female had knee, arm and foot joint pain associated with arthritis. Daily application of a cream prepared as in Example 4 to the affected areas provided relief from pain.

### Example 13

### Pain Study

A 62 year old female had knee pain associated with strenuous exercise. Application of a cream prepared as in Example 4 to the joint area provided pain relief.

### Example 14

### Dermal and Transdermal Cream Formulations

Additional cream formulations for dermal and transdermal vehicles according to the present invention were also investigated.

TABLE 5
Psoriasis Study Creams

| Component - by weight percentage | (MA) | (SA) | (SAMA) |
|---|---|---|---|
| Part A | | | |
| Olive oil PEG 7 | 0.7 | 0.7 | 0.7 |
| Evening Primrose Oil | 1.1 | 1.1 | 1.1 |
| Biovera Oil | 2.2 | 2.2 | 2.2 |
| Jojoba Wax | 2.2 | 2.2 | 2.2 |
| TEGO Soft M (isopropyl myristate) | 1.5 | 1.5 | 1.5 |
| TEGO Soft CT (caprylic/capric acid) | 1.5 | 1.5 | 1.5 |
| Squalene | 1.9 | 1.9 | 1.9 |
| CHREMOPHOR RH 40 | 0.4 | 0.4 | 0.4 |
| SOLULAN 98 | 0.7 | 0.7 | 0.7 |
| Cocoa Butter | 3 | 3.0 | 3.0 |
| Shea Butter | 1.5 | 1.5 | 1.5 |
| Olive Butter | 0.7 | 0.7 | 0.7 |
| Cetearath-20 | 1.1 | 1.1 | 1.1 |
| TEGO Acid S 40 P | 1.1 | 1.1 | 1.1 |
| Glycerol monostearate | 1.1 | 1.1 | 1.1 |
| Stearic Acid | 11.1 | 11.1 | 11.1 |
| Part B | | | |
| Water | to 100% | to 100% | to 100% |
| EDTA | 0.04 | 0.04 | 0.04 |
| Tetrasodium EDTA | 0.04 | 0.04 | 0.04 |
| Propylene Glycol | 3 | 3 | 3 |
| Dimethylaminoethanol | 1.5 | 1.5 | 1.5 |
| Part C | | | |
| Dimethicone, 50 cts | 0.7 | 0.6 | 0.7 |
| Cyclomethicone | 1.5 | 1.5 | 1.5 |
| Vitamin E-acetate | 0.7 | 0.7 | 0.7 |
| Vitamin E | 0.4 | 0.4 | 0.4 |
| Part D | | | |
| PHENONIP | 0.4 | 0.4 | 0.4 |
| GERMALL PLUS | 0.4 | 0.4 | 0.4 |
| COSMOPERINE | 0.9 | 0.9 | 0.9 |
| Meglumine hydrochloride | 4.5 | 0 | 1.9 |
| Arginine hydrochloride | 2.8 | 0 | 2.8 |
| Meglumine | 0 | 0 | 2.6 |
| Salicylic Acid | 0 | 1.9 | 1.9 |
| Part E | | | |
| NATIPIDE mix | 9.7 | 7.8¹ | 9.7 |
| (8 parts NATIPIDE II mixed with | | | |
| 1 part meglumine hydrochloride | | | |
| and 1 part arginine hydrochloride, | | | |
| unless otherwise indicated) | | | |

Part A was heated to 70 degrees. Part B was heated to 70 degrees and blended with Part A. When the mixture cooled to 50 degrees, part C components were added and then Part D ingredients were added one at a time, with homogenization after each addition. Part E was added using an overhead stirrer.
¹NATIPIDE Mix is 100% NATIPIDE II

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A dermally-acting composition for application to the skin, said composition comprising at least one delivery vehicle and meglumine.

2. The composition of claim 1, wherein said delivery vehicle is selected from the group consisting of a liposome, an nanoparticle, and a phospholipid.

3. A dermally-acting composition for application to the skin, said composition comprising a liposome component and meglumine.

4. The composition of claim 3, wherein said meglumine is a hydrochloride salt.

5. The composition of claim 3, further comprising at least one compound selected from the group consisting of arginine and salicylic acid.

6. The composition of claim 5, wherein said arginine is a hydrochloride salt.

7. The composition of claim 3, wherein said formulation further comprises a penetration-enhancing compound.

8. The composition of claim 4, further comprising salicylic acid.

9. The composition of claim 2, wherein said liposome component is selected from the group consisting of NATIPIDE II, BIPHASIX, and NANOSOMES.

10. The composition of claim 2, wherein said delivery vehicle is selected from the group consisting of NATIPIDE II, BIPHASIX, NANOSOMES, PHOSAL, and PHOSPHOLIPON.

11. A dermally-acting composition for application to the skin, said composition comprising a delivery vehicle, meglumine, and at least one additional substance selected from the group consisting of water, oil, wax, squalene, myristate, triglycerides, cocoa butter, shea butter, alcohol, stearate, a chelating agent, propylene glycol, SEPIGEL, silicone, a silicone derivative, a vitamin, and an amino acid.

12. The dermally-acting composition of claim 11, wherein said delivery vehicle comprises a liposome component.

13. A dermally-acting composition for application to the skin, said composition comprising 0.01%-35% delivery vehicle and 0.001%-30% meglumine.

14. The composition of claim 13, wherein said delivery vehicle comprises a liposome component.

15. A dermally-acting composition for application to the skin, said composition comprising 0.01%-35% delivery vehicle, 0.001%-30% meglumine, and 0%-30% arginine.

16. The composition of claim 15, wherein said delivery vehicle comprises a liposome component.

17. A dermally-acting composition for application to the skin, said composition comprising:
a) 0%-5% Meglumine Hydrochloride;
b) 0%-5% Arginine hydrochloride;
c) 0%-5% SEPIGEL 305;
d) 0.01%-30% NATIPIDE mix; and
e) sufficient water to adjust the weight of the composition to 100% of a preselected value;
wherein said NATIPIDE mix consists of 0-20% meglumine hydrochloride, 0-20% arginine hydrochloride, with the remainder of the NATIPIDE mix being NATIPIDE II.

18. A dermally-acting composition for application to the skin, said composition comprising:
a) 0%-4.5% Meglumine Hydrochloride;
b) 0%-2.8% Arginine hydrochloride;
c) 0.01%-9.7% NATIPIDE mix;
d) 0%-2.6% meglumine;
e) 0%-1.9% salicylic acid; and
f) sufficient water to adjust the weight of the composition to 100% of a preselected value;
wherein said NATIPIDE mix is selected from the group consisting of:
1) Eight parts NATIPIDE II mixed with one part meglumine hydrochloride and one part arginine hydrochloride; and
2) NATIPIDE II.

19. A dermally-acting composition for application to the skin, said composition comprising a delivery vehicle and at least one compound selected from the group consisting of meglumine hydrochloride and a hydrochloride salt of a derivative of meglumine.

20. A dermally-acting composition for application to the skin, said composition comprising a liposome and at least one compound selected from the group consisting of meglumine hydrochloride and a hydrochloride salt of a derivative of meglumine.

21. The composition of claim 20, said composition further comprising at least one compound selected from the group consisting of arginine hydrochloride and a hydrochloride salt of a derivative of arginine.

22. The composition of claim 20, said composition further comprising salicylic acid.

23. The composition of claim 21, said composition further comprising salicylic acid.

24. A method for reducing the level of 3-deoxyglucosone (3DG) in the skin of a mammal, said method comprising contacting the skin of said mammal with a dermally-acting composition, said composition comprising a delivery vehicle and meglumine, thereby reducing the 3DG level in the skin of said mammal.

25. The method of claim 24, wherein said delivery vehicle comprises a lipid component.

26. The method of claim 24, wherein said dermally-acting composition further comprises arginine.

27. The method of claim 24, wherein said dermally-acting composition further comprises salicylic acid.

28. The method of claim 26, wherein said dermally-acting composition further comprises salicylic acid.

29. A method for reducing the level of 3-deoxyglucosone (3DG) in the skin of a mammal, said method comprising contacting the skin of said mammal with a dermally-acting composition, said composition comprising a liposome component and meglumine, thereby reducing the 3DG level in the skin of said mammal, wherein said method is used to treat a skin condition selected from the group consisting of skin aging and skin wrinkling.

30. A method for reducing the level of 3-deoxyglucosone (3DG) in the skin of a mammal, said method comprising contacting the skin of said mammal with a dermally-acting composition, said composition comprising a liposome component and meglumine, thereby reducing the 3DG level in the skin of said mammal, wherein said method is used to prevent a skin condition selected from the group consisting of skin aging and skin wrinkling.

31. A method for reducing the level of 3-deoxyglucosone (3DG) in the skin of a mammal, said method comprising contacting the skin of said mammal with a dermally-acting composition, said composition comprising a liposome component and meglumine, thereby reducing the 3DG level in the skin of said mammal, wherein said method is used to treat pain.

32. A method for reducing the level of 3-deoxyglucosone (3DG) in the skin of a mammal, said method comprising contacting the skin of said mammal with a dermally-acting composition, said composition comprising a liposome component and meglumine, thereby reducing the 3DG level in the skin of said mammal, wherein said method is used to treat at least one inflammatory disorder.

33. The method of claim 32, wherein said inflammatory disorder is selected from the group consisting of eczema, psoriasis, rosacea, and radiation-induced dermatitis.

34. A method for reducing the level of 3-deoxyglucosone (3DG) in the skin of a mammal, said method comprising contacting the skin of said mammal with a dermally-acting composition, said composition comprising a liposome component and meglumine, thereby reducing the 3DG level in the skin of said mammal, wherein said method is used to treat itch in a mammal.

35. The method of claim 34, wherein said itch is the result of a condition selected from the group consisting of cutaneous itch, neuropathic itch, neurogenic itch, mixed-type itch, and psychogenic itch.

36. A method for reducing the level of 3-deoxyglucosone (3DG) in the skin of a mammal, said method comprising contacting the skin of said mammal with a dermally-acting composition, said composition comprising a delivery vehicle and meglumine, thereby reducing the 3DG level in the skin of said mammal, wherein said method is used to treat a condition selected from the group consisting of skin aging, skin wrinkling, inflammation, itch and pain.

37. A method for reducing the level of 3-deoxyglucosone (3DG) in the skin of a mammal, said method comprising contacting the skin of said mammal with a dermally-acting composition, said composition comprising a delivery vehicle and meglumine, thereby reducing the 3DG level in the skin of said mammal, wherein said method is used to prevent a skin condition selected from the group consisting of skin aging, skin wrinkling, and inflammation.

38. A kit for reducing the level of 3-deoxyglucosone (3DG) in the skin of a mammal, said kit comprising:
a) a dermally-acting composition for application to the skin, said composition comprising a delivery vehicle and meglumine;
b) an applicator; and
c) instructions for the use of the kit.

39. A kit for reducing the level of 3-deoxyglucosone (3DG) in the skin of a mammal, said kit comprising:
a) a dermally-acting composition for application to the skin, said composition comprising a liposome component and meglumine;
b) an applicator; and
c) instructions for the use of the kit.

40. A kit for reducing the level of 3-deoxyglucosone (3DG) in the skin of a mammal, said kit comprising:
a) a dermally-acting composition for application to the skin, said composition comprising a liposome component, meglumine, and at least one compound selected from the group consisting of arginine and salicylic acid;
b) an applicator; and
c) instructions for the use of the kit.
